(19) United States
(12) Patent Application Publication
(10) Pub. No.: US 2004/0086495 A1
(43) Pub. Date: May 6, 2004

(54) Method for the treatment of arteriosclerosis

(75) Inventors: Josef Kriegelstein, Colbe (DE); Susanne Klumpp, Munster (DE)

Correspondence Address: SUGHRUE MION, PLLC, 2100 PENNSYLVANIA AVENUE, N.W., SUITE 800, WASHINGTON, DC, 20037, US

(73) Assignee: EUCRO EUROPEAN CONTRACT RESEARCH GMBH & CO. KG

(21) Appl. No.: 10/189,188

(22) Filed: Jul. 5, 2002

(30) Foreign Application Priority Data
Jun. 13, 2002 (DE) 102 26 420.1

(51) Int. Cl. A61K 48/00

(52) U.S. Cl. 424/93.21

(57) ABSTRACT

The invention refers to a method for the prevention of arteriosclerosis or the reduction of the arteriosclerosis risk, by purposefully effecting the phosphorylation status of BAD.

The invention refers to a method for treating arteriosclerosis. It claims the priority of German patent application 102 26 420.1, which is referred to for the purpose of disclosure.

Cardiovascular diseases like cardiac infarction, stroke and embolism are among the most common causes of death in the industrialized nations. These diseases are often caused by arteriosclerosis. Arteriosclerosis is understood as a morphological symptom complex of changes on the inner vessel walls, comprising local accumulations of fat, complex carbohydrates, blood components as well as fibrous tissue and calcium combined with changes on the medium vessel wall. These factors lead to bulged and sclerotic regions in the artery wall.

The development of arteriosclerotic damages (lesions) especially comprises three processes, namely the migration, proliferation and accumulation of macrophages, T-lymphocytes and smooth muscle cells in the inner vessel wall, the production of extracellular matrix across the smooth muscle cells as well as the accumulation of lipids in macrophages, muscle cells and the extracellular matrix (Ross, R. (1993). The pathogenesis of atheroclerosis: a perspective for the 1990s. Nature 1993 Apr. 29; 362 (6423) 801-809).

The genesis of arteriosclerotic lesions is initiated by an injury or dysfunction of the endothelian tissue. This inter alia initiates a thrombocyst adhesion and -aggregation and due to this a secretion of growth factors like e.g. PDGF (Platelet derived growth factor). This is followed by a proliferation of intimal smooth muscle cells and in the end—by the production of a new inner vessel wall (neointima)—a thickening of the vessel wall. Moreover endothelian lesions cause an increased permeability of the vessel wall, besides increasing expression of adhesion molecules leading to an invasion of macrophages and leucocytes and, in consequence, to a subsequent inflammatory reaction.

Besides smoking, high blood pressure and diabetes mellitus, an increased level of blood lipids—especially an increased level of blood cholesterol exceeding 200 mg/day—represents an important risk factor for the genesis of arteriosclerosis. Blood lipids are composed of neutral fats (triglycerides), phospholipids, cholesterol, cholesterol esters and free fatty acids. In the blood, they are not present in the free form, but as so-called lipoproteins, i.e. bound to carrier proteins. These carrier proteins, according to their properties in ultra-centrifugation or electrophoresis, are subdivided into several types, namely into chylomicrons, very-low-density-lipoproteins (VLDL), intermediate-density-lipoproteins, low-density-lipoproteins (LDL) and high density lipoproteins (HDL); they are summarized as apo-lipoproteins. The essential component of LDL is cholesterol.

High LDL-concentrations lead to an increased deposit of cholesterol at the vessel wall, since LDL transports cholesterol to the peripheral cells. Thereby they promote the genesis of arteriosclerosis. In contrast, elevated HDL-levels display a protective function.

The prominent arteriosclerotic effect of LDL is also caused by reactive oxygen species or enzymes released from the surrounding tissues, which can oxidize LDL to oxLDL. Infiltrating macrophages can intake oxLDL by their scavenger receptors and—as a consequence of the intracellular lipid accumulation—become so-called foam cells, secreting pro-inflammatory, prothrombotic and atherogenic substances as well as releasing cytokines followed by a proliferation of vascular smooth muscle cells (de Winter M. P. et al., Macrophage scavenger receptor class A: A multifunctional receptor in arteriosclerosis. Arterioscler Thromb Vasc Biol. 2000 February; 20 (2):290-297).

The originally small areas of LDL deposit progressively accumulate to form a plaque. By additional calcium deposits plaques can be formed, which massively contract the vessel. In consequence of a plaque rupture, a thrombus may arise, leading to an embolism and in consequence to ischemia in the surrounding tissue and possibly to an ischemic attack.

Since an increased blood lipid level is considered to be a major risk factor for arteriosclerosis, known therapeutic concepts for treatment and prevention are based on reducing the increased plasma lipid level, thereby especially focusing on a reduction of LDL and its precursor VLDL. To achieve this aim it is known to employ fibrato- or nicotinic acid derivatives, which reduce the concentration of plasma triglycerides and plasma cholesterol by diminution of synthesis and increased decomposition of potentially atherogenic VLDL. Also ion exchange resins can be used to reduce the concentration of potentially atherogenic LDLs.

Another therapeutic approach for the treatment of arteriosclerosis tries to reduce the LDL-concentration by inhibitors of HMG-CoA-reductase consituting the key enzyme of cholesterol biosynthesis and by stimulating an overexpression of liver LDL-receptors. These HMG-CoA-reductase inhibitors, also designated “statines”, can reach a diminution of the potentially atherogenic LDL-cholesterol of up to 60%.

Furthermore, it is known that apoptotic processes take part in the genesis of arteriosclerosis. Apoptosis (programmed cell death) is the elimination of cells as a reaction to stress factors or toxic substances or as a consequence of a purposeful cellular suicidal reaction. Apoptosis is achieved by a targeted activation of signal transduction pathways, leading to characteristic DNA-fragmentation, a rupture of the nuclear membrane, chromatin condensation, shrinking of the nucleus and membrane blabbing (Kerr J. F. et al (1972). Apoptosis: a basic biological phenomenon with wide-ranging implications in tissue kinetics. Br J Cancer 26, 1972 Aug.; 26 (4): 239-257; Searle J. et al. (1982) Necrosis and apoptosis: distinct models of cell death with fundamentally different significance. Pathol Annu 17, 229-259). The elimination of dead cells by phagocytosis is performed by neighboring cells.

Several signal transduction pathways are described for the induction of apoptosis. In mammals, apoptosis is mainly regulated by caspases, Apaf1 (apoptotic protease-activating factor) and the Bcl2 family (see Adams J M et al (1998). The Bcl-2 protein family: arbiters of cell survival. Science 1998 Aug. 28; 281 (5381): 1322-1326; Ashkenazi et al (1998). Death receptors: signaling and modulation. Science 1998 Aug. 28; 281 (5381): 1305-1308).

Several investigations on human atherectomies detect apoptosis in arteriosclerotic plaques (Han et al (1995). Evidence for apoptosis in human atherogenesis and in rat vascular injury model. Am J Pathol 1995 Aug.; 147 (2), 267-277; Hegyi, L. et al (1996). Foam cell apoptosis and the development of the lipid core of human atherosclerosis. J. Pathol 1996 December; 180 (4), 423-429). Thus, apoptotic processes were revealed in macrophages and smooth muscle cells. It is further known, that after a plaque rupture, apoptosis mainly takes place at the fibrous cap, at the rupture position, near the fat inclusions and around the necrotic center (Crisby et al (1997). Cell death in human atherosclerotic plaques involves both oncosis and apoptosis. Artherosclerosis 1997 Apr.; 130 (1-2), 17-27).

Many clues indicate, that the destruction of the endothel by apoptosis constitutes an initial event in the development of arteriosclerosis, since pro-arteriosclerotic factors like oxLDL, angiotensin II or reactive oxygen in vitro also induce apoptosis in endothelian cells (Dimmler et al (1997). Angiotensin II induces apoptosis of human endothelian cells. Protective effects of nitric oxide. Circ Res. 81, 970-976; Dimmler et al (1997). Suppression of apoptosis by nitric oxide via inhibition of ICE-like and CPP32-like proteases. J. Exp. Med. 185, 601-608; Hermann et al (1997). Shear stress inhibits H₂O₂ induced apoptosis of human endothelial cells by modulation of the glutathione redox cycle and nitric oxide synthase. Arterioscler Thromb Vasc Biol 1997 Dec.; 17 (12) 3588-3592). The endothelian apoptosis disrupts the integrity of the endothelian layer and thereby contributes to the pro-inflammatory reaction which leads to the development of plaques. Therefore, apoptosis in the endothelian cells is essential for the process of plaque-erosion and thrombocyte aggregation.

Further, the role of NO as an important physiological mediator in apoptosis signal-transduction is discussed. Former studies showed that NO can induce apoptosis in different cell types. More recent studies in contrast point to anti-apoptotic effects and a protection of endothelian cells by NO (Dimmler et al (1997). Angiotensin II induces apoptosis of human endothelian cells. Protective effects of nitric oxide. Circ Res. 81, 970-976; Dimmier et al (1997). Suppression of apoptosis by nitric oxide via inhibition of ICE-like and CPP32-like proteases. J. Exp. Med. 185, 601-608; Hermann et al (1997). Shear stress inhibits H₂O₂ induced apoptosis of human endothelial cells by modulation of the glutathione redox cycle and nitric oxide synthase. Arterioscler Thromb Vasc Biol 1997 Dec.; 17 (12) 3588-3592). According to this, endothelian cells protect themselves against apoptosis by releasing nitric oxide inhibiting the enzymes catalyzing programmed cell death. Oxygen radicals however inactivate nitric oxide and thereby maintain the potential to induce apoptosis. Thus, endothelian cells die even in the presence of NO. Although the dead cells are replaced by novel cells, the latter show a reduced capacity to release NO. The diminished production of nitric oxide in parallel favors the progression of arteriosclerosis. The reduced production of nitric oxide further supports the proliferation of smooth vascular muscle cells, thus further constricting the blood vessels and leading to the known consequences of oxygen depletion. Also this mechanism points to a fundamental relevance of endothelian cells with respect to the genesis of arteriosclerosis.

Inhibiting apoptosis in endothelian cells therefore is in the focus of research for an effective prevention and therapy for arteriosclerosis. For some time, attempts have therefore been made to use the insight into correlations between apoptosis of endothelian cells and arteriosclerosis—especially the knowledge about the arteriosclerosis inducing factors—for the development of a therapeutic substance. Nevertheless these attempts remained to be unsuccessful so far, even though facts about the contribution of BAD (Bcl-2/Bcl-X_{L}-Antagonist causing cell death) are available.

BAD belongs to a group of arteriosclerosis regulating proteins designated as the Bcl-2 family, comprising pro-apoptotic proteins like Bax and anti-apoptotic proteins like Bcl-2 and Bcl-X. BAD selectively forms dimers with Bcl-2 and Bcl-X (Yang E., et al. (1995) BAD, a heterodimeric partner for Bcl-X_{L} and Bcl-2 displaces Bax and promotes cell death. Cell 1995 Jan. 27; Vol. 80 (2) 285-291) and in consequence replaces Bax as a binding partner of Bcl-X_{L}. The anti-apoptotic effects of Bcl-2/Bcl-X_{L} are thus blocked. Besides of this, the released Bax induces apoptotic reactions (Korsmeyer S J. (1999). BCL-2 gene family and the regulation of programmed cell death. Cancer Res 1999 Apr.1; 1:59 (7 suppl) 1693-1700). Probably this reaction has to be regarded as the essential mechanism, by which apoptosis is promoted.

BAD can be present in several distinct phosphorylated forms. It has been known for some time, that serine¹¹² as well as serine¹³⁶ can be phosphorylated, which has been prooven in vivo. It is also known, that the removal of the phosphate groups is catalyzed by PP1 (protein phosphatase 1) and PP2B (protein phosphatase typ 2 B) (Ayllon V. et al. (2000) Proteinphosphatase 1 α is a ras-activated BAD phosphatase that regulates interleukin-2 deprivation-induced apoptosis. EMBO J. 2000 May 15; Vol. 19 (10) 2237-2246; Wang H G et al., (1999) Ca²⁺ induced apoptosis through calcineurin dephosphorylation of BAD. Science 1999 Apr. 9; Vol. 284 (5412) 339-343). Furthermore it has been found recently, that BAD can also be phosphorylated at serine¹⁵⁵. The phosphorylation at this serine in vitro is accomplished by Proteinkinase A (PKA). This enzyme is activated by cAMP.

If BAD is present in the phosphorylated state (in the following designated as “phospho-BAD”), the binding to Bcl-2/Bcl-X becomes impossible and phospho-BAD dissociates. Phospho-BAD in contrast binds to the so-called 14-3-3 protein. The free Bcl-X_{L} can display its anti-apoptotic effect and interact with Bax (Zha J. et al., (1996) Serine phosphorylation of death agonist BAD in response to survival factor results in binding to 14-3-3 not Bcl-X_{L}, Cell 1996 Nov. 15; Vol. 87 (4) 619-628). In consequence, the phosphorylation of BAD favors anti-apoptotic processes (Lizcano J. M., Morice N. and Cohen P. (2000) Regulation of BAD by cAMP dependent protein kinase is mediated via phosphorylation of a novel side, Ser¹⁵⁵ . Biochem J. 2000 Jul. 15; Vol.: 349 (Pt 2): 547-557; Tan Y. et al (2000) BAD Ser155 Phosphorylation regulates BAD/BclX_{L} interaction and cell survival. J. Biol. Chem. 2000 Aug. 18; 275 (33): 25865 - 25869).

This mechanism probably is the base for the well-known phenomenon of HDL displaying anti-atherogenic properties, as it was shown, that HDL can protect endothelian cells against apoptosis by means of inducing the proteinkinase Akt. This proteinkinase can also be induced by the lysophospholipids isolated from HDL-particles (Nofer et al. (2001) Suppression of endothelian cell apoptosis by high density lipoproteins (HDL) and HDL-associated lysosphingolipids. J. Biol Chem 2001 Sep. 14; 276 (37): 34480-5).

The established therapies for arteriosclerosis commonly interfere with the plasma lipid level, especially by substances decreasing the concentration of blood lipids. This is also valid for approaches involving a repression of apoptosis in endothelian cells (e.g. by increasing HDL). They also primarily focus on regulating the blood lipid level.

Based on this, it is an object of the present invention to provide methods as well as a substance or a substance group to directly or indirectly interfere with the arteriosclerosis promoting apoptosis of endothelian cells.

The invention is based on the idea of utilizing and supporting these anti-apoptotic effects for regulation of the arteriosclerosis-promoting apoptosis in a targeted and purposeful manner by stabilizing BAD mainly in its anti-apoptotic phospho-form or by transforming it into this form. This can basically be accomplished by directly or indirectly blocking the dephosphorylation of phospho-BAD as well as by directly or indirectly stimulating and inducing phosphorylation.

According to a first embodiment of the present invention, repressing or reducing dephosphorylation is accomplished by preferably administering therapeutic substances, which inhibit serine-threonine-phosphatases, especially the type 2 C protein phosphatases (PP2C).

Therapeutic substances according to the before described characteristics refer to all biologically or pharmaceutically active substances, which can directly or indirectly lead to a diminution or reduction of the expression of these proteases or their biological activity. This definition therefore also comprises oligonucleotide-therapeutics like antisense-oligonucleotides.

The protein phosphatase type 2C (PP2C) belongs to the eucaryotic serine-threonine-phosphatases (PP1, PP2A, PP2B, PP2C) with a dephosphorylating activity being dependent on Mn²⁺ or Mg²⁺ ions (McGowan, C H. and Cohen P. (1988) Protein phosphatase-2C from rabbit skeletal muscle and liver: an Mg²⁺⁻dependent enzyme. Meth. Enzymol. 1988; Vol. 159, 416-426). PP2C takes part in regulating apoptosis. The overexpression of this enzyme is lethal (Wenk J. and Mieskes G. (1995) Cytosolic and nuclear localization of protein phosphatase 2C beta 1 in COS and BHK cells. Eur. J. Cell Biol 1995 December; 68 (4), 377-386); Cheng A. et al (1999) Dephosphorylation of cyclin-dependent kinases by type 2C protein phosphatases. Genes Dev. 1999 Nov. 15; 13 (22), 2946-2957). There is no confirmed information yet about the exact mechanism, by which PP2C interferes with apoptotic regulation.

PP2C has so far been detected in several tissues like e.g. neuronal tissue. In contrast it has so far not been detected in endothelian cells. In consequence, the regulation of apoptosis controlled by phospho-BAD and PP2C in these cells was unknown. For this reason, there were—until now—no therapeutic approaches for the treatment or prevention of arteriosclerosis starting from regulating the concentration of BAD or phospho-BAD.

It has now been succeeded also to detect PP2C besides BAD in endothelian cells and to show that phospho-BAD is a substrate for PP2C. It is therefore favorable to administer a therapeutic especially inhibiting PP2C (as a serine-threonine-phosphatase) and by this to purposefully repress the dephosphorylation of phospho-BAD. By this an effective therapeutic approach for the treatment and prevention of arteriosclerosis is provided.

It is a known property of PP2C to be stimulated by unsaturated fatty acids (Klumpp, S., Selke, D., Hermes Mayer J. (1998) Protein phosphatase 2C active at physiological Mg²⁺: stimulation by unsaturated fatty acids. FEBS letters 1998 Oct. 23; 437 (3): 229-232). This is relevant for the reason of fatty acids being present in endothelian cells in high concentrations and thereby being able to induce apoptosis in endothelian cells by stimulating PP2C, in consequence leading to an increased risk of arteriosclerosis. Since fatty acids can induce the apoptosis of endothelian cells by stimulating PP2C activity, it is a special advantage of the invention to base the therapy and prevention of arteriosclerosis also on inhibiting the dephosphorylation of phospho-BAD by endothelian phosphatase PP2C being activated by fatty acids. This can—according to the invention—be accomplished by a targeted suppression of fatty acid activation.

According to a further embodiment of the present invention, an increase of the phospho-BAD ratio can also be accomplished by a substance causing an increased expression, induction, activation or stimulation of BAD phosphorylating protein kinases. Preferred kinases of this kind are e.g. the protein kinase B (PKB or Akt), the mitogen-activated protein (MAP) kinase-activated protein kinase-1 (MAPKAP-K1 or RSK) and the protein kinase A. These kinases phosphorylate Serine-112, Ser-136 or Ser-155 of BAD. The stimulation of BAD phosphorylation according to the invention can e.g. be accomplished by a substance interfering in a regulatory way with the signal transduction or expression processes and thereby causing an increase of kinase activity or by a direct activator of the BAD phosphorylating kinases.

The invention therefore provides an effective approach for a therapeutic treatment and prevention of arteriosclerosis, since controlling the present amount of active pro-apototic BAD—according to the invention by means of influencing the state of phosphorylation—especially counteracts the initial event of arteriosclerosis development, namely the apoptosis of endothelian cells.

In a preferred embodiment of the invention a high level of phospho-BAD and in consequence a low level of BAD is maintained by means of preventing or at least reducing the dephosphorylation by phoshatases, especially by PP2C. This can be achieved in several ways, e.g. by the application of:

a) a phospatase-inhibitor

b) a substance binding to phospho-BAD, stabilizing it and/or, if needed, making it inaccessible for phosphatases

c) an antisense-oligonucleotide repressing expression of a selected phosphatase, especially of PP2C, in endothelian cells

It was shown to be especially advantageous to achieve the inhibition of phospho-BAD dephosphorylation according to the invention by antisense-oligonucleotides and especially by employing them to complex the mRNA of a PP2C. The oligonucleotides may be of synthetic as well as of natural origin. They can e.g. be isolated from yeast.

The invention provides methods allowing to identify suitable inhibitors of phosphatases or binding partners of phospho-BAD, which can—according to the invention—be employed to inhibit dephosphorylation.

According to one embodiment, an active substance can be identified and provided, which inhibits PP2C and in consequence the dephosphorylation of phospho-BAD. This method is based on the in vitro incubation of PP2C with phospho-BAD in the presence of a test compound, followed by the subsequent test of phospho-BAD dephosphorylation being reduced or inhibited.

Using this type of identification system substances can be found, which can be employed to treat or prevent arteriosclerosis according to the invention. A special advantage is based on the fact, that the regulatory effect of phospho-BAD on apoptosis indirectly is also dependent on PP2C activity.

According to another preferred aspect of the method according to the invention, at least one fatty acid, having the property to activate PP2C, is added during the incubation step. By this experimental approach especially those substances can be identified which selectively inhibit activation of PP2C by fatty acids. Thus, a preferred method is provided to identify an active substance especially inhibiting endothelian apoptosis at atherogenic fatty acid concentrations, i.e. the identified substance selectively counteracts this effect of increased fatty acid levels. This high selectivity is especially advantageous, since otherwise undesired side effects caused by impediment of further regulatory processes including PP2C effects may occur. An unscheduled inhibition of apoptosis might e.g. lead to an increased risk of cancer development, since programmed cell death is a major mechanism to regulate cell proliferation. Selectively inhibiting fatty acid activation of PP2C favorably reduces the expected side effects of a therapeutic substance, since only apoptosis induced or stimulated by atherogenic factors would be reduced or prevented.

According to a further embodiment a method is proposed to identify a substance to regulate the described apoptotic processes, in which method—after inducing apoptosis in endothelian cells—an enzymatically active cellular extract is prepared, that is subsequently incubated with phospho-BAD and one or several test substances. Afterwards a possible inhibition of phospho-BAD dephosphorylation by the test substance is evaluated.

This method is especially favorable, since it considers the physiological characteristics of the endothelian cells and in consequence enables the identification of test substances especially suitable for the therapeutic approach according to the invention. This method can alternatively be performed with the substances being directly tested in an endothelian cell culture instead of a cellular extract. The evaluation of apoptosis induction in the endothelian cells can be performed according to the methods described in example 2.

Apoptosis can be preferably induced by at least one PP2C activating fatty acid. This method as described above has the advantage to provide a selectively acting substance.

A further embodiment provides a method to identify a substance, which comprises the screening of a substance library with PP2C as a target in order to identify binding partners. The identified substances are then incubated with PP2C and phospho-BAD and their effect on the dephosphorylation of phospho-BAD is analyzed. This can be initially performed in a cell-free system followed by subsequent testing of the candidates on endothelian cells.

This coincides with the advantage, that only those substances are tested for PP2C-modulating activity, that already have shown to bind to PP2C in the previous screen. This favorably reduces the number of possible test substances and thereby strongly increases the efficiency of the method. As substance libraries, chemical, natural, biological or peptide-libraries can be chosen according to the invention. A large number of such libraries is known as technical standard and can be employed in connection with the invention. For identification of potentially active peptides, phage- and phagemid-libraries are especially suitable.

Furthermore, a method is provided to screen a substance library with phospho-BAD as a target in order to identify binding partners. Instead of phospho-BAD, also phosphorylated peptides corresponding to the respective phosphorylated regions at serine 112, 136 and 155 can be used as targets. This has the advantage, that selectively those binding partners are isolated, that bind to the relevant phosphorylated BAD-sites and not to other structures of phospho-BAD. Possible peptide targets for the identification of potentially active substances are:

| | |
|---|---|
| 112-site: | |
| R(107)SRMSS(112)YPDRG(117) | SEQ ID No. 1 |
| 136-site: | |
| R(132)GSRS(136)APPNN(141) | SEQ ID No. 2 |
| 155-site: | |
| R(149)ELRRMS(155)DEFEGS(161) | SEQ ID No. 3 |

These sequences are antigen sequences from the mouse. Instead, alternatively the respective human BAD-peptide sequences can be used. Obviously the selected peptide sequence can also be expanded or shifted in N- or C-terminal direction.

Also here the identified ligands can subsequently be incubated with PP2C and phospho-BAD followed by an evaluation of their effect on phospho-BAD dephosphorylation.

This embodiment has the advantage, that the assay selectively enables the detection of those substances binding to phosphorylated BAD and making the phosphorylation site inaccessible for PP2C. In this way, suitable inhibitors of the dephosphorylation step can be identified, which do not directly act on PP2C. Preferably the activity of PP2C and in consequence the regulation of other metabolic pathways, in which PP2C is involved as a regulator, is not influenced. In this respect, it is advantageous to screen peptide libraries (or phagemid-libraries), since peptide candidates display the interactions typical for proteins and peptides and therefore have a probability to display specific binding. Furthermore, the peptide candidates' binding specificity to phospho-BAD can be purposefully increased by several screening steps and recombination of suitable candidates.

Additionally, a method for producing a therapeutic for the treatment of arteriosclerosis is provided, in which in a first step a substance with inhibitory effect to the respective apoptotic processes in endothelian cells is identified, preferably by a method according to the invention. In a further step this substance is produced in a sufficient amount and mixed with a physiologically acceptable carrier substance. A therapeutically or pharmacologically active substance is provided, which can be employed for the treatment of arteriosclerosis.

According to an advantageous further aspect of the above mentioned method, PP2C is selected from a group consisting of PP2Cα, PP2Cβ, PP2Cγ, PP2Cδ and their subtypes. These subtypes are described in the following publications, which are hereby fully incorporated by reference (Terasava et al. (1993) Molecular cloning of a novel isotyp of Mg²⁺-dependent protein phosphatase β enriched in brain and heart. Arch. Biochem. Biophys. 307, 342-349; Hou et al., (1994) Molecular cloning of cDNAs encoding to isoforms of protein phosphatase 2Cβ from mouse testis, Biochem. and Mol. Biol. Int. 32, 773-780; Kato et al., (1995) Molecular cloning and expression of mouse Mg dependent protein phosphatase typ 2Cβ-4. Arch. Biochem. 318, 387-393). Preferably PP2Cα and/or PP2Cβ, especially PP2α is used as PP2C.

According to the invention phospho-BAD, that is phosphorylated at the positions serine^{155,} serine¹¹² and/or serine¹³⁶ can be used. In respect to all of these phosphorylation sites it is known, that they prevent the interaction of BAD with Bcl-2 and Bcl-XL. On the other hand, phosphorylation of serine¹³⁶ promotes the binding of BAD to the 14-3-3 protein. As an especially advantageous variant, BAD with a phosphorylated serine¹⁵⁵ is used. Phosphorylation at serine¹⁵⁵ prevents the binding of BAD to Bcl-X_{L} and is thus responsible for the regulatory effect. Here it could be shown, that PP2C preferably dephosphorylates P-ser¹⁵⁵ compared to P-ser¹¹² and P-ser¹³⁶. This selectivity underlines the essential role of PP2C in regulating apoptosis. For reason of this selectivity, it is especially advantageous to identify an inhibitor effectively suppressing dephosphorylation at serine¹⁵⁵, mainly for reason of the pivotal role of serine¹⁵⁵ in preventing apoptosis. As a further variant, BAD phosphorylated at serine¹⁷⁰ can be used.

According to a favorable further aspect of the above described method, the phospho-BAD is radioactively labeled to facilitate the detection. For this, ([³²P]) BAD can be used. As further detection methods, also antibodies directed against phospho-BAD and/or BAD (without influence on the phosphorylation status) can be employed. It is further advantageous to use antibodies selective for P-ser¹¹², P-ser¹³⁶ or P-ser¹⁵⁵. This allows to analyse, if a test substance selectively prevents the dephosphorylation at one of the mentioned positions.

For activating PP2C, preferably fatty acids with a length of at least 15 C-atoms are selected, being at least unsaturated at one position and possessing at least one free carbonic acid group. Examples for selectively PP2C activating fatty acids of this kind are oleic acid, arachidonic acid and gincolic acid. It has been demonstrated that they activate PP2C 10- to 15-fold.

Further, Mn²⁺ and Mg²⁺ are added to the test mixture, preferably Mg²⁺ in a concentration of 0,5 to 1,5 mM. Furthermore, the mixture contains additional additives, like e.g. buffer, further cofactors and/or similar substances.

The depicted methods can be employed in a favorable manner to detect substances, which prevent the dephosphorylation of phospho-BAD by a PP2C and therefore may be used as a therapeutic to treat arteriosclerosis.

It is especially advantageous, if substances employed according to the invention prevent endothelian apoptosis by blocking fatty acid activation of PP2C.

It is further especially preferred if these substances competitively inhibit the binding of activating fatty acids to PP2C. These substances may as well be fatty acids, modified fatty acids or fatty acid analogues blocking the binding site of PP2C for activating fatty acids but not possessing PP2C activating properties themselves.

Inhibitory derivatives of PP2C activating fatty acids may further be found among biphosphonate-fatty acids and/or their derivatives. These substances further have the advantageous property to bind to the calcium deposits in the vessels via their biphosphonat residue and therefore can act more precisely at the desired location. They can be synthesized from the respective fatty acid by phosphonation of the carbonic acid groups. Examples are biphosphonic acids and their salts, preferably according to the following general formula

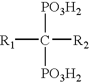

in which R₁ is a linear or branched-chain alkyl residue comprising 1 to 12 carbon atoms, which may be substituted by amino groups, N-mono or di-alkyl-amino-groups, wherein the alkyl-groups can comprise 1 to 12 C-atoms and/or SH-groups or a substituted or unsubstituted carbo- or heterocyclic aryl residue which may possess one or several heteroatoms and as substitutes branched-chain and/or linear alkyl residues with 1 to 12 C-atoms, free or mono-respective dialkylated amino groups with 1 to 6 C-atoms or halogen atoms and wherein R₂ is OH, a halogen atom, preferably Cl, H or NH2. Examples for those substances are ibandronic acid, etidronic acid, clodronic acid, pamidronic acid, aledronic acid, risedronic acid, tiludronic acid, zoledronic acid, cimadronic acid, neridronic acid, olpadronic acid, 3-pyrrol-1-hydroxypropane-1-1-diphosphonic acid and/or minodronic acid, diphosphonate-1-1-dicarbonic acid (DPD) and/or 1-methyl-1-hydroxy-1.1-diphosphonic acid (MDP).

The active substances according to the invention may further be employed for diagnostic purposes (e.g. for localizing plaques) or as contrast media. The respective use of biphosphonate fatty acid derivatives is especially advantageous, since they (as mentioned) bind to calcium deposits via their biphosphonate residues and thereby detect the hot spots of arteriosclerosis (plaques). Thus, they may e.g. be employed in scintigraphic methods. For this purpose, preferably DPD and MDP are employed. Furthermore, the biphosphonate-group can be radioactively labeled by technetium-99-meta. The fatty acid residue in case of receptor binding can then transport the radioactive signal to the binding site consisting of a plaque, thereby enabling diagnosis. Furthermore, the biphosphonate fatty acid derivatives combined with so-called paramagnetic iron-nanoparticles can be used as contrast media in magnetic resonance tomography (MRT). Thereby, plaques can be detected in a preferable way enabling diagnosis of developing or present arteriosclerosis already in an early state before devastating sanitary damages have occurred.

Likewise the substance applicable according to the invention may be a negative regulator for a PP2C displaying fatty acid activation. Such a substance can e.g. bind to the respective regulatory center of PP2C and thereby prevent activation.

According to a further embodiment the inhibitory substance can bind to the active center of PP2C and thereby competitively inhibit the binding of PP2C to phospho-BAD. In this respect it is advantageous, if the substance in its three-dimensional structure essentially corresponds to the three-dimensional structure of phospho-BAD constituting an analogue being not or only slowly convertible. Nevertheless, also competitive inhibitors with different structures may be employed according to the invention. According to a further embodiment antibodies or antibody fragments directed against PP2C can be employed according to the invention. Antibodies or antibody fragments specifically targeted to regulatory sites of PP2C can advantageously block the binding sites of PP2C, thereby preventing the activation of PP2C.

Furthermore, a substance can be used according to the invention, which binds to phospho-BAD and thereby renders the phosphate residue of PP2C inaccessible. Such a substance may e.g. be a peptide or a polypeptide. The use of a polypeptide offers the advantage, that it can be isolated from a peptide library by means of established screening procedures and be also optimized or modified in direction of a high specificity. Such a polypeptide should preferably posses an interaction site like e.g. a binding pocket, by which it can bind to phospho-BAD to render the phosphorylation site inaccessible for PP2C. If this site constitutes a binding pocket, it may e.g. largely correspond to the antigen binding sites of anti-phospho-BAD antibodies in respect to its three-dimensional structure. Since antibodies of this kind are already known, such a substance can be easily generated from the known amino acid sequences of the antigen binding sites. The binding pocket can also be modified in order to reach an especially high specificity for phospho-BAD at its respective phosphorylation sites and to reduce side effects with other cell components if necessary.

In another preferred embodiment according to the invention, the substance's binding pocket in its three-dimensional structure largely corresponds to the antigen binding site of an antibody directed against phospho-BAD serine¹¹², phospho-BAD serine¹³⁶ and phospho-BAD serine¹⁵⁵. In this context it is especially advantageous, to form the binding pocket in correspondence to phospho-BAD serine¹⁵⁵, since it was shown, that PP2C preferably dephosphorylates this site having a special relevance for the regulatory mechanism of BAD.

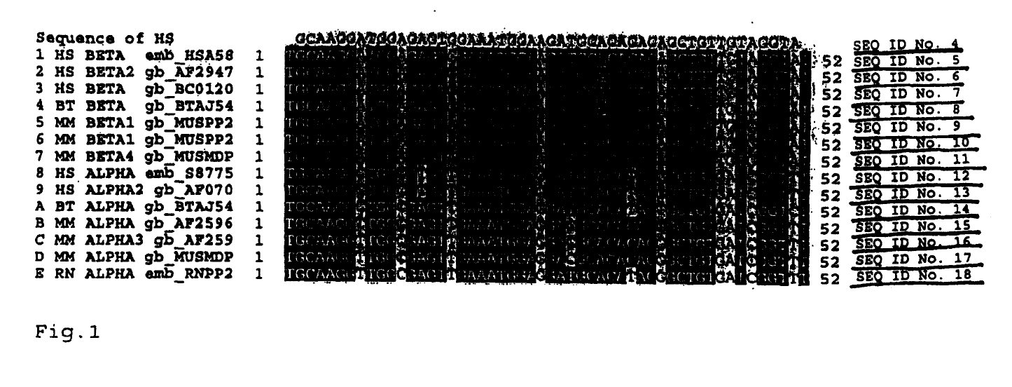

The dephosphorylation of phospho-BAD can also be prevented by suppressing the synthesis of PP2C in the cell. For this aim, a molecule can be employed, which complexes the mRNA of PP2C thereby blocking its translation. This molecule can be an at least partially single-stranded oligonucleotide hybridizing to the mRNA of PP2C (a so-called antisense-oligonucleotide). The mRNA nucleic acid sequences of several PP2C subtypes are accessible in standard internet databases (e.g. those offered by NCBl). Starting from these sequences, established methods allow to design an antisense-molecule hybridizing to the relevant mRNA. Respective examples are listed in FIG. 1.

Preferably the biomolecule complexing the mRNA of PP2C is introduced into endothelian cells to prevent apoptosis. Advantageously, the biomolecule is an oligonucleotide according to the above mentioned embodiments.

The invention further provides a nucleic acid construct expressible in cells, which codes for an expression product able to complex the mRNA of a PP2C. This expression product preferably is a single-stranded oligonucleotide. This can e.g. display the sequence (completely or partially) of the opposite strand of a PP2C mRNA, so that it hybridizes to the PP2C mRNA to form a double helix. Examples of respective oligonucleotides are given in FIG. 1.

According to a favorable development, the nucleic acid construct according to the invention is an expression vector expressible in eucaryots. It is especially favorable, if the coding sequence of PP2C is (at least partially) cloned in inverse direction downstream of an expression vector promoter being expressible in eucaryots. In this way, a transcript is simultaneously generated when the sequence is read, this transcript acting as an antisense-molecule, complexing the mRNA of PP2C and thereby preventing its translation.

This nucleic acid construct can be employed in a method to prevent apoptosis in endothelian cells, wherein an endothelian cell is transfected with a respective nucleic acid construct.

In a further embodiment of the present invention, the phosphorylation of BAD to phospho-BAD can be supported, in consequence keeping the level of pro-apoptotic BAD at a preferably low level. For this aim, it is proposed to directly or indirectly influence the activity or expression of the protein kinases (see above) being relevant for BAD phosphorylation. This can e.g. be achieved by an induction or intensification of enzyme expression or by increasing the activity of these enzymes, e.g. by ligands.

In one respective embodiment, the β-2-sympathomimeticum Clenbuterol is administered for the therapy of arteriosclerosis. The endothelian cells harbor β1 - and β2-receptors, so that Clenbuterol can induce the synthesis of TGF-β1 in these cells. Since it is known, that growth factors lead to a phosphorylation of BAD, Clenbuterol can be used to reduce the pro-apoptotic effects of a high BAD-level.

Additionally, Clenbuterol as a β-sympathomimeticum initiates—similarly like adrenaline or noradrenaline—an increase of the cellular cAMP-level, this acting as an activator stimulating protein kinase A. Thus, Clenbuterol also by this mechanism leads to a decrease of the BAD-level while in parallel increasing phospho-BAD values.

This favorable application is in principle also valid for other active substances leading to a rise of cellular cAMP-levels. This group of substances e.g. comprises inhibitors of phosphodiesterase (PDE) and further β-sympathomimetica, which can be employed according to the invention to increase the amount of phosphorylated BAD in a favorable manner. Examples for PDE-inhibitors are coffeine, theopylline and sildenifil. Further members of this group are known from literature.

Further substances, which can lead to an increase of the phospho-BAD value according to the invention, are those substances increasing blood HDL concentrations as well as bioactive sphingolipids, like e.g. sylophosphorylcholin and lysosulfatid. An increased HDL-concentration and bioreactive sphingolipids result in raised phospho-BAD values by stimulating kinase activities.

All of the mentioned substances, molecules and nucleic acid constructs can be favorably employed to treat arteriosclerosis. According to the invention, furthermore a therapeutic or a pharmacologically active substance is provided, comprising or being constituted by at least one of the previously described substances for arteriosclerosis treatment.

For the preparation of a respective therapeutic, the active substance can further be combined with carriers commonly used for medical preparations. Thus, the substance can be prepared for one of the following application forms like e.g. epicutaneously, buccally, lingually, sublingually, orally, rectally, pulmonarily, conjunctivally, per inhalationem, intracardially, intraarterially, intravenously, intralumbally, intrahectally, intracutaneously, subcutaneously, intramuscularly or intraperitoneally.

For this aim, e.g. solutions, suspensions, emulsions, foams, ointments, pastes, tablets, pills, dragees, suppositories, rectal capsules, jellies, sprays, aerosols, inhalants, drops, solutions for injection, solutions for infusion, implants or liposomal systems can be prepared. For the oligonucleotide therapeutics according to the invention galenic forms are necessary, by which the nucleic acids are transported into the cells. This can e.g. be performed by liposomes or viral or non viral vehicles.

For the preparation of such therapeutic the active substance may further be combined with additional therapeutically active substances. In a composite preparation a substance preventing dephosphorylation of phospho-BAD by PP2C can be combined with a substance stimulating BAD phosphorylation according to the invention. It is further possible to combine these substances alone or in combination with a known substance for lipid diminution or with a substance increasing HDL-concentrations. By means of such a composite preparation, an essential factor of arteriosclerosis (the increased lipid level) as well as the consequences of high lipid levels (apoptosis of endothelian cells) can be simultaneously counteracted.

According to the invention, the in vitro methods are especially understood as techniques including the use of cells, cell cultures, organs or parts thereof, organ systems and parts derived of the human body or of animal bodies. The term in vitro especially comprises artificial systems employing cells, cellular extracts, purified components, homogenates, etc.

The term phospho-BAD, used in connection with the methods according to the invention, also comprises the use of possible phospho-BAD analogues and alternative substrates (e.g. Casein), which are dephosphorylated by PP2C.

The substances applicable according to the invention may, in case of chiral substances, be used as racemates and/or isomers.

The invention should be further depicted by means of the following examples:

### EXAMPLE 1

In one embodiment of the test method according to the invention, the dephosphorylation of phospho-BAD by PP2C is accomplished in vitro with employing recombinant BAD (Lizcano, J. M., Morrice, N. and Cohen, P. (2000) Regulation of BAD by cAMP-dependent protein kinase is mediated via phosphorylation of a novel site, ser^{155.} Biochem.J. Vol:349, 547-557). Phospho-BAD was yielded by a reaction mixture (12 μl) containing 12 μg BAD (as GST-fusion protein; Lizcano et al., 2000), 0.55 μg PKA (Sigma), 1 μl ATP, 15 μCi [γ-³²P] ATP, 5 mM MgCl₂ and 25 mM Tris HCl pH 7.5.The reaction mixture was incubated for 30 min at 37° C.

In an alternative preparation method for phospho-BAD, 3.4 μg GST-BAD in 8 mM MOPS pH 7.2; 10 mM β-glycerophosphate, 2 mM EDTA; 0.4 mM sodium ortho-vanadate; 15 mM MgCl₂; 0.04% 2-mercapto-ethanole; 1 μM ATP plus 15 μCi [γ-³²P] ATP (for radioactive detection) were incubated together with the respective kinases (100 ng PKA, 100 ng PKB or 5 mU RSK) in a total volume of 10 μl for 30 min at 30° C.

The phosphorylation reactions were stopped by the addition of 5 μl Laemmli probe buffer. Radioactively labeled and unlabeled ATP was removed by centrifugation (750×gₘₐₓ for 2 min) in Centri-SEP centrifugation columns (emp Biotech, Berlin) following the incubation to prevent a renewed phosphorylation in the second incubation step (with PP2C). Probes should be treated in the further steps without a timely delay.

In order to yield the PP2C enzyme, a cDNA was expressed in E.coli, followed by a purification in Ni-NTA sepharose. In the present case, the cDNA encoding for PP2Cβ from bovine brain was used (J. Neurosci. Res. 51, 328-338 (1998) Protein phosphatase type 2-C isoenzymes present in vertebrate retinae: purification, characterization and localization in photoreceptors).

The dephosphorylation of phospho-BAD by PP2C is dependent as well on time and concentration. Test mixtures must contain Mg²⁺ or Mn²+ ions, since PP2C depends on the presence of these ions. Preferably, the test mixture contains 0.5 to 1.5 mM/l Mg²⁺. The incubation period can at least be 10 min, but preferably is 30 min. PP2C was added in a concentration of at least 30 ng, preferably in a concentration of more than 300 ng.

The radioactively labeled [³²P]BAD purified by centrifugation was 10-times diluted with 25 mM Tris/HCl pH 7.5. The 15 μl incubation mixtures were incubated at 37° C. (or 30 ° C.) for 30 min. The test mixtures contained: 9 μl [³²P]BAD (0.2 μg); 1.5 μl 10-times reaction buffer (200 mM Tris/HCl pH 7.5; 100 mM MgCl₂, 10% glycerol, 1% 2-mercaptoethanole); 300 ng PP2C.

The reaction was stopped by the addition of 5 μl denaturating buffer and kept on ice until the proteins were separated on a 12.5% SDS PAGE mini-jelly. The degree of dephosphorylation or phosphorylation was analyzed by autoradiography.

According to a further possible in vitro test mixture, the activity of PP2C is analyzed by incubating a 30 μl mixture containing 20 mM Tris-HCl pH 7.5; 0,01% 2-mercaptoethanole; 1.3 mg/ml BSA and 1 μM [³²P] casein (5×10⁴ cpm) at 30° C. for 10 min (McGowan and Cohen, 1988; Cohen 1991). To determine PP2C activity in the presence of fatty acids, 0.7 μM magnesium acetate were added. The reaction was stopped by the addition of 200 μl 20% trichloric acid. After centrifugation at 10000 g (5 min), 200 μl of the supernatant was analyzed for its concentration of [³²P] phosphate.

The respective results allow to examine if the test system is functional. The substances to be tested for their capacity to inhibit the dephosphorylation are added to a respective test mixture and incubated as described above. By means of autoradiography it is tested in the following to what extent an inhibition—compared to the controls (without test substance)—has taken place. Preferably the potential active substance is tested at different concentrations to avoid losing a promising candidate not displaying an effect at the wrong concentration. Candidates, especially promising ones, can also be tested for their synergistic effects in one common mixture.

In order to test to what extent a targeted inhibition of a fatty acid activated PP2C is possible, a PP2C activating fatty acid was also added to the mixture. As an example, oleic acid was used preferably in concentrations between 10 and 500 μM. By respective controls it can be determined, how far the test substance only stops the fatty acid activation without in parallel inhibiting the “regular” (non stimulated) activity of PP2C. By such experimental approach, those substances can be selectively determined, which precisely and solely inhibit fatty acid induced apoptosis without interfering with normal regulatory processes.

The test substances identified this way, can in the following be tested in endothelian cells (see example 2.)

### EXAMPLE 2

Endothelian cell cultures were suspended and the test cell cultures were inocculated at an initial cell density of 4×10⁴ cells/cm² on poly-L-lysine coated bulbs or poly-L-lysine coated cover-glasses, placed in petri dishes for TUNEL assays. The cultures were kept at 37° C. temperature and 5% CO₂ in a humidified atmosphere in DMEM, whereby 20% of foetal calf serum and antibiotics were added. The medium was changed every 2 days.

4 days after inocculation, the cultures were exposed to a serum free medium for 24 h. In the following, the cells were treated with the test substances and/or PP2C activating fatty acids, fatty acid derivatives or binding agents. Fatty acids and/or their derivatives were first dissolved in pure DMSO and then diluted in culture medium leading to a final concentration of 0.016% DMSO in all experiments.

In order to induce apoptosis, preferably the PP2C activating fatty acids (like e.g. oleic or gincolic acid) were added, although also other known apoptosis-inductors can be used. In order to detect PP2C in endothelian cell culture, 20 mM of Mg²+ ions were employed. Subsequent to the incubation it was analyzed, to what extend the test substances were able to inhibit apoptosis.

For apoptosis detection, the nuclear form and chromosomal structure can be visualized by the application of Hoechst 33258 as a dye for nuclear DNA. The cultures are incubated with 10 μg/ml Hoechst 33258 in methanol for 10 min, followed by washing with methanol and PBS. Subsequently, the nuclear morphology was analyzed under a fluorescence microscope. The number of endothelian cells with shrunken nuclei and condensed chromatin were counted in three areas in every of the four different culture bulbs. The results were expressed and analyzed as the percentage of endothelian cells with condensed chromatin and shrunken nuclei in the total of cells.

For the detection of DNA fragmentation, TUNEL assays were performed, wherein a commercially available kit was used according to the manufacturer's instructions. Cell monolayers were briefly fixed in methanol at −20° C. for 20 min and then incubated at 37° C. for 1 h with digoxigenin labeled ddUTP in the presence of a terminal transferase. The reaction was stopped and anti-digoxigenin-antibodies, coupled to fluorescin isothiocyanate, were added and the mixture incubated for further 30 min. Subsequently, the fluorescence was analyzed with a suitable microscope (LSM 510, Zeiss, Germany).

The activity of caspase-3 was determined with a fluorimetric kit. This assay is based on a hydrolysis of the peptide substrate acetyl-asp-glu-val-asp-7-amido4-methylcoumarin (Ac-DEVD-AMC) by caspase-3, leading to a release of the fluorescent AMC component. The cells were harvested and pelleted by centrifugation at 600× g at 4° C. for 5 min. The supernatant was withdrawn and the pellet washed with PBS. In the following, the pellet was dissolved in lysis buffer containing 50 mM HEPES, pH 7.4; 5 mM 3-(3 cholamidopropyl)dimethylammonium 1-propanesulfonat and 5 mM dithiothreitol and incubated on ice for 20 min. Subsequently the cells were centrifuged at 14 000× g at 4° C. for 15 min. The supernatant was mixed with the reaction buffer (10 mM Ac-DEVD-AMC; 20 mM HEPES, pH 7.5; 2 mM EDTA; 0,1% 3-(3-cholamidopropyl)dimethylammonio-1-propanesulfonat and 5 mM dithiothreitol). The fluorescence of AMC was measured at an activating wavelength of 360 nm and an emission wavelength of 460 nm. The results were calculated in relation to an AMC standard curve and preferably determined as nmol AMC/mg protein/min. The amount of protein was measured by using the “bicincolinic acid protein assay” kit with bovine serum albumin as a standard.

By means of the described methods it can be determined, if the treated cells show apoptotic processes. Thereby, test substances can be determined, which reduce or prevent apoptosis in endothelian cells. In this way, valuable potential therapeutics for arteriosclerosis can be identified.

### REFERENCE

Adams J M et al (1998). The Bcl-2 protein family: arbiters of cell survival. Science 1998 Aug. 28; 281 (5381): 1322-1326.

Ashkenazi et al (1998). Death receptors: signaling and modulation. Science 1998 Aug. 28; 281 (5381): 1305-1308.

Ayllon V. et al. (2000) Proteinphosphatase 1α is a ras-activated BAD phosphatase that regulates interleukin-2 deprivation-induced apoptosis. EMBO J. 2000 May 15; Vol. 19 (10) 2237-2246.

Cheng A. et al (1999) Dephosphorylation of cyclin-dependent kinases by type 2C protein phosphatases. Genes Dev. 1999 Nov. 15; 13 (22), 2946-2957.

Crisby et al (1997). Cell death in human atherosclerotic plaques involves both oncosis and apoptosis. Artherosclerosis 1997 Apr.; 130 (1-2), 17-27.

de Winter M. P. et al., Macrophage scavenger receptor class A: A multifunctional receptor in arteriosclerosis. Arterioscler Thromb Vasc Biol. 2000 February; 20 (2):290-297.

Dimmler et al (1997). Angiotensin II induces apoptosis of human endothelian cells. Protective effects of nitric oxide. Circ Res. 81, 970-976.

Dimmier et al (1997). Suppression of apoptosis by nitric oxide via inhibition of ICE-like and CPP32-like proteases. J. Exp. Med. 185, 601-608.

Han et al (1995). Evidence for apoptosis in human atherogenesis and in rat vascular injury model. Am J Pathol 1995 Aug.; 147 (2), 267-277.

Hegyi, L. et al (1996). Foam cell apoptosis and the development of the lipid core of human atherosclerosis. J. Pathol 1996 December; 180 (4), 423429.

Hermann et al (1997). Shear stress inhibits H₂O₂ induced apoptosis of human endothelial cells by modulation of the glutathione redox cycle and nitric oxide synthase. Arterioscler Thromb Vasc Biol 1997 December; 17 (12) 3588-3592.

Hou et al., (1994) Molecular cloning of cDNAs encoding to isoforms of protein phosphatase 2Cβ from mouse testis, Biochem. and Mol. Biol. Int. 32, 773-780.

J. Neurosci. Res. 51, 328-338 (1998) Protein phosphatase type 2-C isoenzymes present in vertebrate retinae: purification, characterization and localization in photoreceptors.

Kato et al., (1995) Molecular cloning and expression of mouse Mg dependent protein phosphatase typ 2Cβ-4. Arch. Biochem. 318, 387-393.

Kerr J. F. et al (1972). Apoptosis: a basic biological phenomenon with wide-ranging implications in tissue kinetics. Br J Cancer 26, 1972 August; 26 (4): 239-257.

Klumpp, S., Selke, D., Hermes Mayer J. (1998) Protein phosphatase 2C active at physiological Mg²⁺: stimulation by unsaturated fatty acids. FEBS letters 1998 Oct. 23; 437 (3): 229-232.

Korsmeyer S J. (1999). BCL-2 gene family and the regulation of programmed cell death. Cancer Res 1999 Apr. 1; 1:59 (7 suppl) 1693-1700.

Lizcano J. M., Morice N. and Cohen P. (2000) Regulation of BAD by cAMP dependent protein kinase is mediated via phosphorylation of a novel side, Ser⁵⁵ . Biochem J. 2000 Jul. 15; Vol.: 349 (Pt 2): 547-557.

McGowan, C H. and Cohen P. (1988) Protein phosphatase-2C from rabbit skeletal muscle and liver: an Mg²⁺-dependent enzyme. Meth. Enzymol. 1988; Vol. 159, 416-426.

Nofer et al. (2001) Suppression of endothelian cell apoptosis by high density lipoproteins (HDL) and HDL-associated lysosphingolipids. J. Biol Chem 2001 Sep. 14; 276 (37): 34480-5.

Ross, R. (1993). The pathogenesis of atheroclerosis: a perspective for the 1990s. Nature 1993 Apr. 29; 362 (6423) 801-809.

Searle J. et al. (1982) Necrosis and apoptosis: distinct models of cell death with fundamentally different significance. Pathol Annu 17, 229-259.

Tan Y. et al (2000) BAD Ser155 Phosphorylation regulates BAD/BclX_{L} interaction and cell survival. J. Biol. Chem. 2000 Aug. 18; 275 (33): 25865 - 25869.

Terasava et al. (1993) Molecular cloning of a novel isotyp of Mg²⁺-dependent protein phosphatase β enriched in brain and heart. Arch. Biochem. Biophys. 307, 342-349.

Wang H G et al., (1999) Ca²⁺ induced apoptosis through calcineurin dephosphorylation of BAD. Science 1999 Apr. 9; Vol. 284 (5412) 339-343.

Wenk J. and Mieskes G. (1995) Cytosolic and nuclear localization of protein phosphatase 2C beta 1 in COS and BHK cells. Eur. J. Cell Biol 1995 December; 68 (4), 377-386).

Yang E., et al. (1995) BAD, a heterodimeric partner for Bcl-X_{L} and Bcl-2 displaces Bax and promotes cell death. Cell 1995 Jan. 27; Vol. 80 (2) 285-291.

Zha J. et al., (1996) Serine phosphorylation of death agonist BAD in response to survival factor results in binding to 14-3-3 not Bcl-X_{L}, Cell 1996 Nov. 15; Vol. 87 (4) 619-628.

1. Method for the prevention of arteriosclerosis or reduction of the arteriosclerosis risk, characterized in a prevention or reduction of the dephosphorylation of phospho-BAD in endothelian cells.

2. Method for the prevention of arteriosclerosis or reduction of the arteriosclerosis risk, characterized in an increase of the phosphorylation of BAD in endothelian cells.

3. Method according to one of the above mentioned claims, characterized in the use of an antisense-oligonucleotide.

4. Method according to one of the above mentioned claim, characterized in the use of a PP2C mRNA antisense-oligonucleotide.

5. Method according to one of claim 1 or 2, characterized in influencing the phosphorylation/dephosphorylation of ser¹¹², ser¹³⁸ and/or ser¹⁵⁵ of BAD.

6. Method according to one of claims 1, 2 or 5 characterized in the use of a serine-threonine-phosphatase-inhibitor.

7. Method according to claim 6, characterized in the use of a PP2C inhibitor.

8. Method according to claim 2, characterized in the use of β-sympathomimetica, β-2-sympathomimetica, in particular Clenbuterol and/or phosphodiesterase inhibitors.

9. Method for the identification of a substance, which inhibits the dephosphorylation of phospho-BAD by PP2C, characterized in
a. Incubating PP2C with phospho-BAD or a convertible analogue and at least one test substance in vitro,
b. Detecting the effectiveness of the test substance with respect to the dephosphorylation of phospho-BAD by a PP2C.

10. Method according to claim 9, characterized in adding in step a) at least one fatty acid activating PP2C.

11. Method for the identification of a substance, which can affect the apoptotic processes in endothelian cells by inhibiting the dephosphorylation of phospho-BAD by PP2C, characterized in
a. Inducing apoptosis in endothelian cells,
b. Contacting and incubating with one or several test substances,
c. Detecting the effectiveness of th test substance with respect to the dephosphorylation of phospho-BAD or a convertible analogue by a PP2C.

12. Method according to claim 11, characterized in that after step a) an enzymatically active cellular tract is obtained from the induced endothelian cells.

13. Method according to one or more of the above mentioned claims, characterized in that for the induction in step a) at least one PP2C activating fatty acid is used, which preferably has a length of at least 15 C-atoms, is unsaturated in at least one position and possesses at least one free carbonic acid group and is preferably oleic acid, arachidonic acid and/or gincolic acid.

14. Method for the identification of a substance, which inhibits the dephosphorylation of phospho-BAD by a PP2C, characterized in
a. Screening a substance library with a PP2C or a PP2C-fragment as a target for the identification of binding partners
b. Incubating the substances isolated in step a) with PP2C and phospho-BAD or a convertible analogue
c. Detecting the effectiveness of the substances isolated in step a) on the dephosphorylation of phospho-BAD or the convertible analogue.

15. Method according to claim 14, characterized in that the substance library is selected from a group of chemical, natural, biological or peptide libraries.

16. Method for the identification of a substance, which inhibits phospho-BAD dephosphorylation by a PP2C, characterized in
a. Screening a substance library with phospho-BAD or a phosphorylated peptide fragment of BAD as a target for the identification of binding partners interacting with phospho-BAD,
b. Incubating the substances isolated in step a) with PP2C and phospho-BAD or a convertible analogue,
c. Detecting the effectiveness with respect to the dephosphorylation of phospho-BAD or the convertible analogue by PP2C.

17. Method for the production of a therapeutic for the treatment of arteriosclerosis, characterized in
a. Identifying a substance,regulating the apoptotic processes in endothelian cells by inhibiting the dephosphorylation of phospho-BAD by PP2C by a method according to at least one of the claims 9, 11, 14, 16,
b. Mixing this substance with at least one physiologically acceptable carrier substance to form a therapeutic.

18. Method according to one or more of the above mentioned claims, characterized in that PP2C is selected from a group comprising PP2Cα; PP2Cβ; PP2Cγ, PP2Cδ and their subtypes.

19. Method according to one or more of the above mentioned claims, characterized in that antibodies against phospho-BAD and/or BAD are used in order to detect the turn-over of phospho-BAD.

20. Method according to one or more of the above mentioned claims, characterized in that phospho-BAD or phospho-BAD peptide fragments are used, which are phosphorylated at the positions serine¹⁵⁵, serine¹¹² and/or serine¹³⁶.

21. Method according to claim 20, characterized in that the peptide fragments are used, which are derived from BAD in the amino acid region surrounding ser¹¹², ser¹³⁶, ser¹⁵⁵.

22. Method according to claim 21, characterized in that the peptide fragments have a length of at least 8 amino acids.

23. Use of a substance generated by a method according to one or more of the above mentioned claims as a therapeutic for the treatment of arteriosclerosis, wherein said substance prevents the dephosphorylation of phospho-BAD by a PP2C.

24. Substance for the inhibition of apoptosis in endothelian cells, characterized in that it is an inhibitor of a PP2C.

25. Substance according to claim 24, characterized in that its is an inhibitor of a PP2C being activatable by a fatty acid.

26. Substance according to claim 24 of 25, characterized in that it is a selective inhibitor of fatty acid activation of PP2C.

27. Substance according tone or more of the claims 24 to 26, characterized in that it competitively inhibits the binding of fatty acids to PP2C.

28. Substance according to one or more of the claims 24 to 27, characterized in that it is a fatty acid, a modified fatty acid, a fatty acid derivative, a biphosphonate fatty acid derivative or a fatty acid analogue.

29. Substance according to claim 28, characterized in that it is a biphosphonic acid or a derivative of such an acid according to the general formula 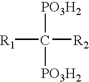
wherein
R₁
is a linear or branched-chain alkyl residue with 1 to 12 carbon atoms, which may be substituted by substitutes such as amino groups, N-mono- or dialkylamino groups, in which the alkyl residues can comprise 1 to 12 C-atoms and/or SH-groups, or
is a substituted or unsubstituted carbo- or heterocyclic aryl residue, that can possibly comprise one or several heteroatoms and as a substitute branched-chain or linear alkyl residues with 1 to 12 C-atoms, free or mono-respective dialkylated amino groups with 1 to 6 C-atoms or halogen atoms and
R₂
is OH, a halogen atom, referably Cl, H or NH₂

30. Substance according to claim 1, characterized in that it is selected from the group comprising ibandronic acid, etidronic acid, clodronic acid, pamidronic acid, alendronic acid, risedronic acid, tiludronic acid, zoledronic acid, cimadronic acid, neridronic acid, olpadronic acid, 3-pyrrol-1-hydroxypropane-1-1-diphosphonic acid and/or minodronic acid, diphosphonate-1,-1-dicarbonic acid (D 0) and/or 1- methyl-1-hydroxy-1.1 -diphosphonic acid (MDP).

31. Substance according to one or more of the above mentioned claims, characterized in that it is a negative regulator of a PP2C being activatable by fatty acids.

32. Substance according to one or more of the/above mentioned claims, characterized in that it binds to the active center of PP2C and/or competitively prevents the binding of phospho-BAD to PP2C.

33. Substance according to claim 32, characterized in that it has a three-dimensional structure essentially corresponding to the three-dimensional structure of phospho-BAD and that it is a substrate analogue not or only slowly convertible.

34. Substance for inhibiting apoptosis in endothelian cells, characterized in that it binds to phospho-BAD and renders the phosphate residue, in particular P-Ser55, inaccessible for a PP2C.

35. Substance according to claim 34, characterized in that it is polypeptide.

36. Substance according to claim 34 or 35, characterized in that it binds to phospho-BAD by means of an interacting region, preferably by a binding pocket.

37. Substance according to claim 36, characterized in that the binding pocket in its three-dimensional structure essentially corresponds to the antigen binding site of antibodies directed against phospho-BAD.

38. Substance according to claim 36 or 37, characterized in that it comprises a binding pocket corresponding to a modified antigen binding site of an anti-phospho-BAD antibody.

39. Substance according to one or more of the claims 36 to 38, characterized in that the binding pocket in its three-dimensional structure essentially corresponds to the antigen-binding site of an antibody directed against phospho-BAD serine¹¹² phospho-BAD serine¹³⁶ and/or phospho-BAD¹⁵⁵.

40. Substance for inhibiting apoptosis in endothelian cells, characterized in that it is a polypeptide binding to a PP2C.

41. Substance according to claim 40, characterized in that it is an antibody or an antibody fragment.

42. Biomolecule, characterized in that it complexes the mRNA of a PP2C.

43. Biomolecule according to claim 42 characterized In that it is an oligonucleotide being at least partially single-stranded.

44. Oligonucleotide according to claim 43, characterized in that it is a nucleic acid sequence, which completely or partially corresponds to the opposite strand of a PP2C mRNA, so that this molecule hybridizes the PP2C mRNA, thereby forming at least parts with double-stranded regions.

45. Use of an oligonucleotide according to claim 43 or 44 as an antisense-oligonucleotide to complex the PP2C mRNA.

46. Method to prevent apoptosis in endothelian cells, characterized in that a substance according to one of the above mentioned claims, especially an at least partially single-stranded oligonucleotide according to claim 44 is introduced into an endothelian cell.

47. Nucleic acid construct expressible in cells, characterized in that the expression product is biomolecule which complexes the PP2C mRNA.

48. Nucleic acid construct according to claim 47, characterized in that the expression product is a single-stranded oligonucleotide.

49. Nucleic acid construct according to claim 47 of 48, characterized in that the expression product is an oligonucleotide with a nucleic acid sequence according to one of the sequences shown in FIG. 1.

50. Nucleic acid construct according to on or more of the claims 47 to 49, characterized in that it is an expression vector expressible in eucaryotic cells.

51. Nucleic acid construct according to one or more of the claims 47 to 50, characterized in that the coding sequence of PP2C is cloned at least partially in inverted orientation behind the promoter of an expression vector expressible in eucaryots.

52. Method to prevent apoptosis in endothelian cells to treat arteriosclerosis, characterized in that an endothelian cell is transfected with a nucleic acid construct according to claims 47 to 51.

53. Use of at least one substance, a biomolecule or a nucleic acid construct according to at least one of the above mentioned claims for the treatment of arteriosclerosis and/or the diseases related thereto.

54. Pharmacologically active composition, comprising at least one substance according to at least one of the above mentioned claims for the treatment of arteriosclerosis.

55. Composition according to claim 54, characterized in that it contains at least a lipid reducing agent and/or an agent increasing the HDL-concentration.

56. Use of β-sympathomimetica, β-2-sympathomimetica, in particular Clenbuterol and/or phosphodiesterase inhibitors to increase the content of phospho-BAD in an endothelian cell for the prevention of apoptosis.
